(19) United States
(12) Patent Application Publication
(10) Pub. No.: US 2015/0353705 A1
(43) Pub. Date: Dec. 10, 2015

(54) PROCESS FOR RECYCLING POLYESTER WASTE

(71) Applicant: Reliance Industries Limited, Mumbai (IN)

(72) Inventors: Gurudatt Krishnamurthy, Bangalore (IN); Anil Krishna Kelkar, Belapur (IN); Anil Kumar Satapathy, Dist. Dhenkanal State (IN); Anjan Kumar Mukhopadhyay, Navi Mumbai (IN); Karunanithi Thandayuthapani, Dist. Raigad (IN); Pushap Sudan, Jammu (Tawi) (IN); Santosh Chandrakant Geedh, Dist. Thane (IN); Venkatachalam Subbiah, Mumbai (IN)

(73) Assignee: Reliance Industries Limited, Mumbai (IN)

(21) Appl. No.: 14/760,283

(22) PCT Filed: Dec. 31, 2013
(86) PCT No.: PCT/IN2013/000821

§ 371 Date: Jul. 10, 2015

(30) Foreign Application Priority Data
Jan. 11, 2013 (IN) 101/MUM/2013

(51) Int. Cl. C08J 11/24; C07C 67/48; C07C 67/03

(52) U.S. Cl. CPC C08J 11/24; C07C 67/03; C07C 67/48; C08J 2367/02

(57) ABSTRACT

The present disclosure relates to a polyester glycolate obtained by glycolyzing a mass of the polyester with excess of ethylene glycol in the presence of an acid catalyst. The present disclosure also relates to a process for manufacturing recycled polyester from the polyester glycolate.

### FIELD OF THE DISCLOSURE

The present disclosure relates to a polyester glycolate composition and a process for preparation thereof. The present disclosure further relates to a process for recycling polyester to produce recycled polyester having improved color values and whiteness index.

### BACKGROUND

Polyethylene terephthalate is a thermoplastic resin possessing excellent characteristic features such as heat resistivity, process-ability, transparency and non-toxicity. Polyethylene terephthalate is one of the versatile engineering plastics used in manufacturing wide range of products such as films, fibers, bottles, container and the like. The rapid development of polyester production industry is inevitably causing the production of industrial polymer waste and post-user waste at large scale. The large scale production of polymer waste and their non-biodegradable nature is posing a biggest threat for the environment. Different approach and methodologies have been adapted in polymer industries to provide a viable solution for handling the polymer waste. The recycling of these polymer wastes is one of the promising methods adapted to control the polymer waste. Further, the consistency in terms of volume and the high scrape value creates an excellent economic environment for the recycling of these polyester wastes.

Many attempts have been made for recycling of these polyester wastes. The process for de-polymerizing the polyester waste and re-polymerizing the de-polymerized product obtained in the de-polymerization step is considered as one of the effective methods. The re-cycled polyester is further used for preparing spinning fibers.

### EXISTING KNOWLEDGE

Different methods and approaches have been adopted for de-polymerizing and re-polymerizing the polyester wastes to obtain recycled polyesters.

The glycolysis of polyester waste is a well-known de-polymerization process. The hitherto reported processes for the glycolysis of polyester wastes disclose the use of metal catalysts such as sodium bicarbonate, zinc acetate, and zinc oxide. The kinetics of these catalysts has been found to be excellent; nevertheless, there have been issues with regard to color of the recycled polyesters and heavy metal content in the final fibers. The fibers produced from the re-cycled polyesters are yellowish in color (poor b* color).

U.S. Pat. Nos. 7,166,690 and 7,511,081, and GB1520426 disclose the use of polybasic acids such as phthalic anhydride, isophthalic acid, terephthalic acid and adipic acid for the de-polymerization of polyester waste. The use of 0.05 to 0.5 wt % dibutyltin oxide (DBTO) as a catalyst during the de-polymerization and re-polymerization is also disclosed. Further, European Patent No. 865464 discloses the de-polymerization of polyester with ethylene glycol at a temperature of 150-300° C. to obtain monomeric and/or oligomericdihydroxy compound such as bis(2-hydroxyethyl)terephthalate (BHET). Conventional trans-esterification catalysts such as salts of Zn, Sb, ti, Sn, Mn or Ge are particularly employed during the de-polymerization process.

Further, U.S. Pat. No. 5,776,989 discloses the use of di-carboxylic acid or di-amine compounds along with glycol ester for the decomposition of cured unsaturated polyester waste. The processes for recycling polyester waste as described in aforementioned prior-art documents only disclose what is expected as per fundamentals of polycondensation polymerization i.e. the use of stoichiometric excess of one kind of di-functional or mono-functional, monomer to depolymerize the polyester alcoholysis or hydrolysis.

### OBJECTS

Some of the objects of the present disclosure are described herein below:

It is an object of the present disclosure to ameliorate one or more problems of the prior art or to at least provide a useful alternative.

It is another object of the present disclosure to provide a process for glycolyzing polyester that completely eliminates the use of metal catalyst.

It is still another object of the present disclosure to provide a polyester glycolate.

It is a yet another object of the present disclosure to provide a process for preparing recycled polyester with enhanced color values and whiteness index.

It is still another object of the present disclosure to provide a process for preparing recycled polyester that eliminates and/or minimizes the use of metal catalysts.

It is a yet another object of the present disclosure to provide recycled polyester having enhanced color value and whiteness index.

It is a further object of the present disclosure is to provide recycled polyester having enhanced color value and whiteness index useful for the production of polyester fibers free from metallic contents.

Other objects and advantages of the present disclosure will be more apparent from the following description when read in conjunction with the accompanying figures, which are not intended to limit the scope of the present disclosure.

### DEFINITIONS

As used in the present disclosure, the following words and phrases are generally intended to have the meanings as set forth below, except to the extent that the context in which they are used to indicate otherwise.

As used the term “polyester” in the context of the present disclosure refer to a mass of recyclable polyester which is worth of being recycled for various reasons wherein the reason for recycling includes the non-limiting examples such as polyester quality upgradation, excess inventory management, managing product recall and the like.

As used the term “Polyester waste” in the context of the present disclosure refers to polyester which is discarded or eliminated for being no longer useful, and subjected to recycling for the purpose of waste and/or inventory management. Particularly, the waste may refer to both ex-factory and in-factory polyester with equal relevance.

As used the terms “polyester” or “polyester waste” or “a mixture of polyester and polyester waste” in the context of the present disclosure may further comprises virgin polyester in any considerably proportion except 100%.

As used the term “virgin polyester” in the context of the present disclosure refer to polyester which is sold without being recycled.

As used the term “Polyester glycolate” in the context of the present disclosure refers to a glycolyzed product obtained from the glycolysis of polyester carried out by using excess of ethylene glycol.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

### SUMMARY

In accordance with one aspect, the present disclosure provides a polyester glycolate composition, said composition comprising:
(A) a polyester glycolate; and
(B) residues of: (i) ethylene glycol and (ii) an acid catalyst.

Typically, the amount of residual ethylene glycol ranges between 0.0002 to 0.0030% by weight of the polyester glycolate composition.

Typically, the amount of residual acid catalyst ranges between 0.0001 to 0.0010% by weight of the polyester glycolate composition.

Typically, the polyester is selected from the group consisting of polyester, polyester waste and a mixture of polyester and polyester waste.

Typically, the polyester waste is at least one selected from the group consisting of fiber waste, hard polymer waste, polymer flakes and combinations thereof.

Typically, the polyester is polyethylene terephthalate.

Typically, the acid catalyst is at least one selected from the group consisting of acetic acid, oxalic acid, trimelletic acid, benzoic acid, propionic acid, butyric acid and tartaric acid.

Typically, the polyester glycolate comprises at least one dihydroxy species selected from the group consisting of monomer, dimer, oligomer or combinations thereof.

Typically, the dihydroxy species is bis-(2-hydroxyethylene) terephthalate.

In accordance with another aspect, the present disclosure provides a process for the preparation of polyester glycolate composition as disclosed in the first aspect of the present disclosure, said process comprising glycolyzing a mass of polyester with excess of ethylene glycol in the presence of an acid catalyst to obtain slurry containing the polyester glycolate composition.

Typically, the method step of glycolyzing comprises refluxing of polyester with excess of ethylene glycol in the presence of an acid catalyst for a time period ranging between 1 to 15 hours, preferably between 5 to 10 hours.

Typically, the proportion of polyester and ethylene glycol ranges between 1:1 and 1:20, preferably 1:1 and 1:10.

Typically, the acid catalyst is at least one selected from the group consisting of acetic acid, oxalic acid, trimelletic acid, benzoic acid, propionic acid, butyric acid and tartaric acid.

Typically, the amount of the acid catalyst is 0.01 to 3.0% of the Mass of the polyester, preferably 0.10 to 1.0%

Typically, the process for the preparation of polyester glycolate composition as disclosed in one of the aspects of the present disclosure further comprises the following steps:
a. filtering the slurry to obtain a mass of polyester glycolate and a filtrate containing a residual ethylene glycol and the acid catalyst; and
b. washing repeatedly the obtained mass, of polyester glycolate with water to remove traces of ethylene glycol and the acid catalyst, if any.

In accordance with still another aspect of the present disclosure, there is provided a recycled polyester being a polycondensation product of a slurry comprising the polyester glycolate composition as disclosed in one of the aspects of the present disclosure, together with a metal catalyst selected from the group consisting of antimony trioxide, titanium alkylates, germanium oxide, tin oxide and antimony acetate.

Typically, the metal residues are not more than 3300 ppm.

In accordance with a yet another aspect of the present disclosure, there is provided a process for manufacturing recycled polyester; said process comprising the following steps;
a. glycolyzing a mass of polyester with an excess of ethylene glycol in the presence of an acid catalyst to obtain a first slurry containing polyester glycolate, a residual ethylene glycol and the acid catalyst;
b. polycondensing the first slurry in the presence of at least one metal catalyst selected from the group consisting of antimony trioxide, titanium alkylates, germanium oxide, tin oxide and antimony acetate to obtain a second slurry containing polyester strands, residual ethylene glycol, and the acid and the metal catalysts; and
c. filtering the second slurry to obtain a recycled polyester.

Typically, the mass of polyester is selected from the group consisting of polyester, polyester waste and a mixture of polyester and polyester waste

Typically, the polyester waste is at least one selected from the group consisting of fiber waste, hard polymer waste, polymer flakes and combinations thereof.

Typically, the process for manufacturing recycled polyester further comprising a method step of washing the recycled polyester stands with excess of water to remove traces of ethylene glycol, if any.

Typically, the method step of polycondensing the first slurry further comprises a method step of incorporating at least one additive selected from the group consisting blue toner, TiO₂, and optical brightener.

Typically, the polyester is polyethylene terephthalate.

Typically, the method step of glycolyzing comprises refluxing of polyester with excess of ethylene glycol for a time period ranging between 1 hr to 15 hrs, preferably 5 hrs to 10 hrs.

Typically, the proportion of polyester and ethylene glycol ranges between 1:1 and 1:20, preferably 1:1 and 1:10.

Typically, the acid catalyst is at least one selected from the group consisting of acetic acid, oxalic acid, trimelletic acid, benzoic acid, propionic acid, butyric acid and tartaric acid.

Typically, the amount of the acid catalyst is 0.01% to 3.0% of the mass of the polyester, preferably 0.10% to 1.0%.

Typically, the polyester glycolate comprises at least one dihydroxy species selected from the group consisting of monomer, dimer, oligomer or combinations thereof.

Typically, the dihydroxy species is bis-(2-hydroxyethylene) terephthalate.

Typically, the recycled polyester comprises metal residues not more than 3300 ppm.

### BRIEF DESCRIPTION OF THE ACCOMPANYING DRAWINGS

FIG. 1 Illustrates the method step of glycolyzation of polyester carried out in the presence of (a) acetic acid catalyst; (b) oxalic acid catalyst; (c) combination of oxalic acid and zinc acetate catalyst; and (d) conventional zinc acetate catalyst.

### DETAILED DESCRIPTION

Accordingly, a process for manufacturing recycled polyester is envisaged in the present disclosure wherein the disadvantages allied with the use of metal catalyst during the conventional recycling processes have been successfully mitigated by the present inventors. The recycled polyester as obtained in accordance with the present disclosure possesses enhanced color values and whiteness index as compared to the recycled polyester obtained by using conventional metal catalysts.

In accordance with one aspect of the present disclosure, there is provided polyester glycolate composition comprising (a) polyester glycolate; and (b) residues of (i) ethylene glycol and (ii) an acid catalyst. The polyester glycolate composition in accordance with the present disclosure is obtained by glycolyzing a mass of polyester with an excess of ethylene glycol in the present of an acid catalyst.

The polyester glycolate composition of the present disclosure comprises residual ethylene glycol in an amount typically ranging between 0.0002 to 0.0030% of the weight of the polyester glycolate composition. The amount of the residual acid catalyst varies between 0.0001 to 0.0010% of the weight of the polyester glycolate composition.

For preparing polyester glycolate of the present disclosure, a mass of polyester is mixed with excess of ethylene glycol and an acid catalyst to obtain a reaction mixture. The reaction mixture is then charged in a reaction vessel and heated to a boiling temperature of ethylene glycol to initiate refluxing. Refluxing is typically continued for a time period ranging between 1 hr and 15 hrs, preferably between 5 hrs and 10 hrs to complete the glycolyzation. The method step of refluxing is typically carried out under nitrogen atmosphere and under constant stirring.

Preferably, after 8 hrs of refluxing, the reaction is stopped and slurry comprising polyester glycolate composition is obtained. The slurry so obtained also comprises residual ethylene glycol and the acid catalyst. After completion of the reaction, the reaction vessel is flushed with nitrogen (0.2 kg/cm²) for a time period of 2 hours. The slurry is then separated from the reaction vessel and subjected to filtration to remove excess of ethylene glycol and to obtain a mass of polyester glycolate which is then repeatedly washed with chilled water to further remove the traces of ethylene glycol. The obtained mass of polyester glycolate is dried under vacuum to obtain polyester glycolate in powder from. The color values of the polyester, glycolate are determined by methods known in the art.

The mass of polyester employed for the purpose of the present disclosure is selected from the group consisting of polyester, polyester waste and a mixture of polyester and polyester waste. In accordance with one of the preferred embodiments of the present disclosure, the mass of polyester is polyester waste. The mass of polyester waste is preferably a mixed waste that comprises a combination of at least two polyester wastes selected from the group consisting of polyester fiber waste, polyester hard waste, and polyester flakes in various weight proportions. The preferred polyester in accordance with the present disclosure is polyethylene terephthalate.

The acid catalyst as employed for the purpose of the present disclosure is selected from the group consisting of acetic acid, oxalic acid, trimelletic acid, benzoic acid, propionic acid, butyric acid, tartaric acid and any combinations thereof. The amount of the acid catalyst is 0.01% to 3.0 wt % of the mass of the polyester, preferably 0.10% to 1.0%.

The proportion of polyester and ethylene glycol ranges between 1:1 and 1:20, preferably 1:1 and 1:10.

The polyester glycolate in accordance with the present disclosure is bis-(2-hydroxyethylene) terephthalate that exists in at least one form selected from the group consisting of monomer, dimer, and oligomers.

The polyester glycolate in accordance with the present disclosure is further characterized for its color value. The use of oxalic acid catalyst improves ‘L’ value by 1 unit and reduces the ‘b’ value by 0.40 unit as compared to the polyester glycolate obtained from the metal catalyzed reaction. Further the obtained value of ‘a’ is close to zero which shows reduction in red undertone. The whiteness index data of the polyester glycolate also confirms the improvement in their whiteness as compared to the polyester glycolate obtained from the metal catalyzed reaction.

The polyester glycolate obtained in the accordance with the process of the present disclosure is further subjected to a polycondensation reaction to obtain recycled polyester.

Therefore, in another aspect, the present disclosure also provides a process for manufacturing recycled polyester having improved color values and whiteness index, said process comprising the following method steps:
(i) glycolyzing a mass of polyester with excess of ethylene glycol in the presence of an acid catalyst to obtain polyester glycolate; and
(ii) polycondensing the polyester glycolate to produce recycled polyester.

The method step of glycolyzing a mass of polyester with excess of ethylene glycol is accomplished in accordance with the process as hereinabove described in the present disclosure for the preparation of polyester glycolate.

In the method step of glycolyzation, the mass of the polyester waste is refluxed with excess of ethylene glycol in the presence of an acid catalyst to obtain first slurry containing polyester glycolate. The proportion of the amounts of polyester and ethylene glycol typically varies between 1:1 and 1:20; preferably 1:1 and 1:10; the preferred range is 1:5.

The acid catalyst used for glycolyzation in accordance with the process of the present disclosure may be an aliphatic or aromatic carboxylic acid. Typically, the acid catalyst is selected from the group consisting of oxalic acid, acetic acid, trimelletic acid, benzoic acid, propionic acid, butyric acid, tartaric acid and any combinations thereof.

The weight proportion of the amount of acid catalyst varies between 0.01 to 3.0%, preferably between 0.10 to 1.0%, with respect to the total mass of the polyester.

The mass of polyester as employed in the method step of glycolyzation is selected from the group consisting of polyester, polyester wastes and mixture of polyester and polyester wastes. In one of the embodiments, the mass of polyester is a polyester waste. Preferably, the polyester waste is a mixed waste that comprises a combination of at least two polyester wastes selected from the group consisting of polyester fiber waste, polyester hard waste and polyester flakes in various weight proportions.

In addition to polyester glycolate, the first slurry also contains residual ethylene glycol and the acid catalyst. The slurry obtained in the method step of glycolyzation is then subjected to a polycondensation reaction by mixing it with a polycondensation catalyst.

The polycondensation catalyst as employed in the process of the present disclosure is a metal catalyst that includes at least one metal catalyst selected from the group consisting of antimony trioxide, titanium alkylates, germanium oxide, tin oxide and antimony acetate.

The use of metal catalysts, particularly antimony trioxide for manufacturing virgin polyesters is well known. During the manufacturing of virgin polyesters, antimony trioxide catalyst remains entrapped in the network of polyester matrix. The presence of entrapped catalyst residues therefore reduces the quality of the virgin resins. Thus, further use of antimony trioxide during recycling of virgin polyester products may therefore augment this problem by raising the proportion of entrapped antimony catalyst in the recycled polymer matrix thereby adversely affecting its quality. To avoid this problem, the present inventors have developed a process for recycling polyesters wherein the use of metal catalyst during the method step of polycondensation is reduced as compared to the conventional processes by using an organic acid catalyst selected from the group consisting of oxalic acid, acetic acid, trimelletic acid, benzoic acid, propionic acid, butyric acid, tartaric acid and any combinations thereof.

In the process of recycling polyester in accordance with the present disclosure, the acid catalyst employed during the method step of glycolyzation is carried forward in the polycondensation step which reduces the requirement of high amount of metal catalyst.

The polycondensation reactor charged with the first slurry and the polycondensation catalyst (metal catalyst) is heated to a temperature varying in the range of 285° C. to 290° C. to initiate the polycondensation process. Typically, the polycondensation is carried out at a temperature of 290° C. and for a time period varying between 100 to 130 minutes to obtain second slurry containing recycled polyester strands, residual ethylene glycol, and the acid and the metal catalysts.

The obtained recycled polyester is then drained as strands and quenched in water.

The process for manufacturing recycled polyester in accordance with the present disclosure may further comprises a method step of washing the recycled polyester strands with excess of chilled water to remove the traces of ethylene glycol, and the traces of acid and metal catalysts, if any.

The recycled polyester obtained in accordance with the process of the present disclosure is of improved color values and whiteness index, and contain metal residues not more than 3300 ppm

One of the preferred polyesters in accordance with the present disclosure is polyethylene terephthalate. The recycled polyester i.e. polyethylene terephthalate obtained in accordance with the process of the present disclosure is of improved color values and obtained in at least one form that comprises chips and fibers. The recycled polyester chips are characterized by having L* values not less than 76.0 and b* values not higher than 2.0, whereas the recycled polyester fiber are characterized by having L* values not less than 88.0 and b* values not higher than 0.1.0.

The color values of recycled polyester chips to a greater extent depend on the thermal behavior of the polyester. Additionally, the hardware dimensions take part in determining the color values of polyester chips. Contrary to the chips, the color values of polyester fibers depend on the fineness of fibers. The values mentioned in the present disclosure for the recycled polyethylene terephthalate is specific for 6 denier fineness fiber.

The recycled polyester prepared in accordance with the process of the present disclosure is further used for manufacturing spinning fibers. The spinning fibers thus obtained contains less amount of metallic residues

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed anywhere before the priority date of this application.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

### Example1

### Lab-Scale Glycolysis of Polyethylene Terephthalate by Using Acetic Acid Catalyst

A four necked glass reactor fitted with a reflux condenser was charged with a mass of polyethylene terephthalate containing 100% (w/w) of polyethylene, terephthalate waste. The polyethylene terephthalate waste was a mixed waste comprising polyethylene terephthalate fibers waste, polyethylene terephthalate hard waste and polyethylene terephthalate flakes in a weight proportion of 89:6.5:4.5, respectively. The mass of polyethylene terephthalate was then mixed with excess of ethylene glycol in a weight proportion of 1:5. The reaction mixture thus obtained was refluxed for 8 hours under continuous stirring in the presence of acetic acid catalyst to obtain a slurry. The refluxing of the reaction mixture was carried out under nitrogen atmosphere. The acetic acid was used in a weight proportion of about 0.5 wt %. After 8 hours of reaction time, the obtained slurry was subjected to filtration to remove excess of ethylene glycol and to obtain a mass of polyester glycolyzate. The obtained mass of polyester glycolyzate was repeatedly washed with chilled water to further remove excess of ethylene glycol and subsequently dried under vacuum to obtain polyester glycolyzate in powdery form.

### Example2

### Lab-Scale Glycolysis of Polyethylene Terephthalate by Using Oxalic Acid Catalyst

This example describes a process for glycolyzing a mass of polyethyleneterephthalate by using oxalic acid catalyst. The process was carried out in the same manner as described in the example-1.

### Example-3

### Lab-Scale Glycolysis of Polyethylene Terephthalate by Using a Combination of Zinc Acetate and Oxalic Acid Catalyst

This example describes a process for glycolyzing a mass of polyethyleneterephthalate by using a combination of zinc acetate and an acid catalyst. 50 ppm of zinc acetate in combination with 0.2 wt % of oxalic acid was used. The reaction was carried out in the same manner as described in the process of example-1.

### Comparative Example-1

### Lab-Scale Glycolysis of Polyethylene Terephthalate by Using a Metal Catalyst

This example describes a process for glycolyzing a mass of polyethylene terephthalate by using zinc acetate catalyst. 105 ppm of zinc acetate was used and the reaction was carried out in the same manner as described in the process of example-1.

Further, the kinetics of the reactions as described in example-1, 2, 3 and comparative example-1 were established and compared. To establish the kinetics of the reactions, the reaction sample was removed from the vessel at the specific intervals and evaluated for intrinsic viscosity (IV). Plot of intrinsic viscosity vs time indicates kinetics of the reaction.

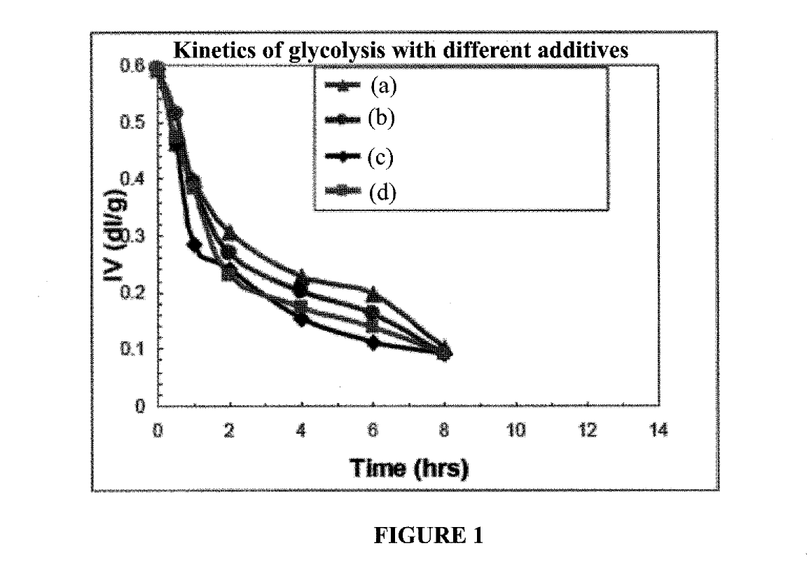

Refer to FIG. 1 of the accompanying drawings for the comparative study of the kinetics of the glycolysis processes. It is evident from the accompanying drawing that kinetic of oxalic acid catalyzed glycolysis reaction matches closely with that of zinc acetate catalyzed reaction. Further, the oxalic acid catalyzed reaction also shows resemblance with the kinetic of glycolysis reaction catalyzed by using the combination of zinc acetate and oxalic acid.

The polyester glycolate obtained in accordance with the processes of example-1, 2, 3 and comparative example-1 is further analyzed for their color values. Their characteristic color values and whiteness index data is tabulated in Table-1.

TABLE 1

| | | Polyester | | | | | | |
|---|---|---|---|---|---|---|---|---|
| | | glycolate | | | | | | |
| | | composition | | | | | Whiteness | |
| | | Ethylene | Acid | | | | index | |
| | | glycol | catalyst | | | | CIE | |
| | | residue | residue | | | | Ganz | Hunter |
| Reaction | Catalyst | (%) | (%) | L* | a* | b* | 82 | lab 60 |
| Example-1 | Acetic acid | 0.0003 | Not | 94.96 | 0.04 | 2.68 | 75.38 | 85.81 |
| | (0.5 wt %) | | detected | | | | | |
| Example-2 | Oxalic acid | 0.0002 | 0.0001 | 95.75 | 0.06 | 2.08 | 80.02 | 88.48 |
| | (0.5 wt %) | | | | | | | |
| Example-3 | Zinc | 0.0003 | 0.0001 | 94.92 | 0.11 | 2.35 | 76.78 | 86.70 |
| | acetate (50 ppm) + | | | | | | | |
| | oxalic acid | | | | | | | |
| | (0.2 wt %) | | | | | | | |
| Comparative | Zinc | 0.0030 | NA | 94.69 | 0.17 | 2.46 | 75.73 | 86.09 |
| Example-1 | acetate | | | | | | | |

It is evident from the provided data that the polyester glycolate obtained from the process of example-2 wherein oxalic acid was used as catalyst shows distinct color values as compared to the polyester glycolate obtained in accordance with processes of example-1, 3 and comparative example-1.

Oxalic acid catalyst shows improved L* by 1 and reduced b* values by 0.38 units respectively, whereas the catalyst comprising the combination of zinc acetate and oxalic acid shows no color change. Further the value of a* closer to zero in case of oxalic acid and acetic acid catalyzed reactions indicates reduction in red undertone. Whiteness index data in case of example-1 and 2 also confirms improvement in the whiteness of the polyester glycolate.

### Example-4

This example describes a process for the recycling of polyethylene terephthalate in pilot scale batch reactor using a modified process to simulate both glycolysis and polycondensation processes.

Monoethylene glycol MEG (22.2 kg) and purified terephthalic acid (PTA) (51.6 kg) (1:1.15 molar ratio) was esterified using oxalic acid (2000 ppm w/w of batch size) as catalyst under nitrogen pressure of 1.7 kg/cm² and at temperature of 260° C. for 300 minutes. The reactor depressurized and 5.64 Kg of, polyester waste was added. The polyethylene terephthalate waste was a combination of polyethylene terephthalate fibers waste, polyethylene terephthalate hard waste and polyethylene terephthalate flakes in a weight proportion of 89:6.5:4.5. Afterwards, the reactor was heated to a temperature of 260° C. to initiate the glycolysis process. The heating of the reaction mixture was carried out under nitrogen atmosphere under continuous stirring.

After 120 minutes of reaction, the slurry was transferred to a polycondensation reactor. In the polycondensation reactor, a combination of antimony tri-oxide (170 ppm w/w of the total batch size) and zinc acetate (80 ppm w/w of the total batch size) catalyst was added. Additional to catalyst, 0.30 ppm of TiO₂ and 15 ppm of blue toners were also added. Afterwards, a vacuum was applied slowly to the polycondensation reactor till a final vacuum of around 1 mm Hg was obtained. The 1 mmHg of vacuum was obtained in 45 min. Followed to this, the temperature was gradually increased to around 285° C. As the reaction progressed, the viscosity increased due to polymerization, hence torque (about 0.5 Nm) and power of the agitator increased. After a reaction time of 130 min, there was a rise in torque. The vacuum was then broken at specified torque value and the reactor was pressurized with nitrogen and recycled polymer was drained as strands and quenched in water bath. The strands were then cut into chips in a pelletizer, which were further dried to remove moisture.

### Example-5

Similar to the process of example-4, the glycolysis of polyethylene terephthalate was carried out by using same amount of oxalic acid catalyst (2000 ppm w/w of batch size) in the glycolyzing step. In this example, the combination of 130 ppm of antimony trioxide and 80 ppm of zinc acetate (80 ppm w/w of batch size) were used as a polycondensation catalyst. The polycondensation of polyester glycolate was carried out in the same manner as described in the process of Example-4.

### Comparative Example-2

This example describes a process for glycolyzing and polycondensing polyethylene terephthalate by using conventional catalysts. Similar to the process of Examples-4&5 the glycolysis of polyethylene terephthalate was carried out by using zinc acetate as catalyst (105 ppm w/w of batch size) in the glycolyzing step. In this example, 250 ppm w/w of batch size, of antimony trioxide was used as a polycondensation catalyst. The polycondensation of polyester glycolate was carried out in the same manner as described in the process of Examples-4&5

The polycondensation time (PC) is an indictor of kinetics of polycondensation reaction. The polycondensation time for the processes of example-4, 5 and comparative example-2 is provided in Table-2.

It is evident from the provided data that the polycondensation time in case of example-4 and 5 wherein combination of antimony trioxide and zinc acetate is used as a polycondensation catalyst is either reduced or closely matches with the polycondensation time of comparative example-2, wherein polycondensation is carried out by using antimony trioxide catalyst as a whole.

The reduced amount of antimony trioxide catalyst during the polycondensation reactions of example-4 and example-5 does not effect the rate of reaction as compared to the polycondensation reaction of comparative example-2, wherein antimony trioxide is used on a stand alone basis. This indicates that the oxalic acid catalyst present in the slurry containing the polyester glycolate also catalyzes the polycondensation reaction of example-4 and example-5.

TABLE 2

| | Experiment No. | | |
|---|---|---|---|
| | Comparative | Experiment | Experiment |
| | example-2 | No. 4 | No. 5 |
| De-polymerization | | | |
| Catalyst, ppm | Zinc acetate, 105 | Oxalic acid, | Oxalic acid, |
| | | 2000 | 2000 |
| Polymerization | | | |
| Catalyst, ppm | Sb₂O₃, 250 | Sb₂O₃ + zinc | Sb₂O₃ + zinc |
| | | acetate, | acetate, |
| | | (170 + 80) | (130 + 80) |
| Blue Toner, ppm | 15 | 15 | 15 |
| TiO₂ | 0.30 | 0.30 | 0.30 |
| Poly indicators | | | |
| PC Time, min | 130 | 112 | 133 |
| Final vacuum | 0.03 | 0.04 | 0.06 |

### TECHNICAL ADVANTAGES

The present disclosure related to a process for recycling polyester waste has the following technical advantages:
(1) Production of recycled polyester with improved/enhanced color values,
(2) Use of non-metallic acid catalyst that produces recycled polyester with lower metallic residues, and
(3) Minimizing the use of metallic catalyst during de-polymerization as well as during re-polymerization process.

The numerical values mentioned for the various physical parameters, dimensions or quantities are only approximations and it is envisaged that the values higher/lower than the numerical values assigned to the parameters, dimensions or quantities fall within the scope of the invention, unless there is a statement in the specification specific to the contrary.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A polyester glycolate composition, said composition comprising:
(A) a polyester glycolate; and
(B) residues of (i) ethylene glycol and (ii) an acid catalyst.

2. The polyester glycolate composition as claimed in claim 1, wherein the residue of ethylene glycol is present in an amount ranging between 0.0002 to 0.0030% by weight of the polyester glycolate composition, and wherein the residue of acid catalyst is present in an amount ranging between 0.0001 to 0.0010% by weight of the polyester glycolate composition.

3. (canceled)

4. The polyester glycolate composition as claimed in claim 1, wherein the polyester is selected from the group consisting of polyester, polyester waste and a mixture of polyester and polyester waste, wherein said polyester waste is at least one selected from the group consisting of fiber waste, hard polymer waste, polymer flakes and combinations thereof, and wherein the polyester is polyethylene terephthalate.

5. (canceled)

6. (canceled)

7. The polyester glycolate composition as claimed in claim 1, wherein the acid catalyst is at least one selected from the group consisting of acetic acid, oxalic acid, trimelletic acid, benzoic acid, propionic acid, butyric acid and tartaric acid.

8. The polyester glycolate composition as claimed in claim 1, wherein the polyester glycolate comprises at least one dihydroxy species selected from the group consisting of monomer, dimer, oligomer or combinations thereof.

9. The polyester glycolate composition as claimed in claim 8, wherein the dihydroxy species is bis-(2-hydroxyethylene)terephthalate.

10. A process for the preparation of polyester glycolate composition of claim 1, said process comprising glycolyzing a mass of polyester with excess of ethylene glycol in the presence of an acid catalyst to obtain a slurry containing the polyester glycolate composition.

11. The process as claimed in claim 10, wherein the method step of glycolyzing comprises refluxing of polyester with excess of ethylene glycol in the presence of an acid catalyst for a time period ranging between 1 to 15 hours.

12. The process as claimed in claim 10, wherein the proportion of polyester and ethylene glycol ranges between 1:1 and 1:20.

13. The process as claimed in claim 10, wherein the acid catalyst is at least one selected from the group consisting of acetic acid, oxalic acid, trimelletic acid, benzoic acid, propionic acid, butyric acid and tartaric acid, wherein the amount of the acid catalyst is 0.01 to 3.0% of the mass of the polyester.

14. (canceled)

15. The process as claimed in claim 10, further comprising the following steps:
a. filtering the slurry to obtain a mass of polyester glycolate and a filtrate containing a residual ethylene glycol and the acid catalyst;
b. washing repeatedly the obtained mass of polyester glycolate with water to remove traces of ethylene glycol and the acid catalyst, if any.

16. A recycled polyester being a polycondensation product of a slurry comprising the polyester glycolate composition of claim 1 together with a metal catalyst selected from the group consisting of antimony trioxide, titanium alkylates, germanium oxide, tin oxide and antimony acetate.

17. The recycled polyester as claimed in claim 16, comprises metal residues not more than 3300 ppm.

18. A process for manufacturing a recycled polyester, said process comprising the following steps:
a. glycolyzing a mass of polyester with an excess of ethylene glycol in the presence of an acid catalyst to obtain a first slurry containing polyester glycolate, a residual ethylene glycol and the acid catalyst;
b. polycondensing the first slurry in the presence of at least one metal catalyst selected from the group consisting of antimony trioxide, titanium alkylates, germanium oxide, tin oxide and antimony acetate to obtain a second slurry containing polyester strands, residual ethylene glycol, and the acid and the metal catalysts; and
c. filtering the second slurry to obtain a recycled polyester.

19. The process as claimed in claim 18, wherein the mass of polyester is selected from the group consisting of polyester, polyester waste and a mixture of polyester and polyester waste, wherein said polyester waste is at least one selected from the group consisting of fiber waste, hard polymer waste, polymer flakes and combinations thereof, and said polyester is polyethylene terephthalate.

20. (canceled)

21. The process as claimed in claim 18, further comprising a method step of washing the recycled polyester strands with excess of water to remove traces of ethylene glycol, if any.

22. The process as claimed in claim 18, wherein the method step of polycondensing the first slurry further comprises a method step of incorporating at least one additive selected from the group consisting blue toner, TiO₂, and optical brightener.

23. (canceled)

24. The process as claimed in claim 18, wherein the method step of glycolyzing comprises refluxing of polyester with excess of ethylene glycol for a time period ranging between 1 hr to 15 hrs.

25. The process as claimed in claim 18, wherein the proportion of polyester and ethylene glycol ranges between 1:1 and 1:20.

26. The process as claimed in claim 18, wherein the acid catalyst is at least one selected from the group consisting of acetic acid, oxalic acid, trimelletic acid, benzoic acid, propionic acid, butyric acid and tartaric acid, and wherein the amount of the acid catalyst is 0.01% to 3.0% of the mass of the polyester.

27. (canceled)

28. (canceled)

29. (canceled)

30. The process as claimed in claim 18, wherein the recycled polyester comprises metal residues not more than 3300 ppm.
